(19) United States
(12) Patent Application Publication
(10) Pub. No.: US 2009/0298197 A1
(43) Pub. Date: Dec. 3, 2009

(54) SERS-BASED METHODS FOR DETECTION OF BIOAGENTS

(75) Inventors: Michael J. Natan, Los Altos, CA (US); Michael Sha, Castro Valley, CA (US); William E. Doering, Mountain View, CA (US)

Correspondence Address: SWANSON & BRATSCHUN, L.L.C., 8210 SOUTHPARK TERRACE, LITTLETON, CO, 80120, US

(73) Assignee: OXONICA MATERIALS INC., Mountain View, CA (US)

(21) Appl. No.: 12/093,748

(22) PCT Filed: Nov. 15, 2006
(86) PCT No.: PCT/US2006/060930

§ 371 Date: Jan. 23, 2009

Related U.S. Application Data
(60) Provisional application No. 60/737,242, filed on Nov. 15, 2005.

(51) Int. Cl. G01N 33/566

(52) U.S. Cl. 436/501

(57) ABSTRACT

An assay and method of assay for optical detection of bioagents, a target nucleic acid or a target protein using a surface enhanced Raman scattering (SERS) active biomolecule molecular beacon. The present invention also provides the assay and method in a multiplexed format.

### FIELD OF THE INVENTION

The invention relates to a bioagent detection system employing SERS (surface-enhanced Raman scattering)-based methods and systems.

### BACKGROUND OF THE INVENTION

The use of fluorescence quenching as a detection method in biological assays is widespread and includes the use of molecular beacons, a technology first described in 1996. Tyagi, S. and Kramer, F. R., “Molecular Beacons: probes that fluoresce upon hybridization” Nature Biotechnol. 1996, 14, 303-308. Molecular beacons typically use a fluorophore reporter dye and a non-fluorescent quencher chromophore. While in close proximity, the fluorophore is quenched by the energy transfer to the non-fluorescent chromophore. However, separating the fluorophore and the quencher results in a fluorescent signal. Molecular beacons have been used in a variety of assay formats, including the monitoring of nucleus activity, the detection of pathogens and SNP detection.

An assay using a fluorescent energy transfer system, such as molecular beacons, does not require the target nucleic acid to be labeled, nor does the target nucleic acid have to be separated from the other components of the assay. For example, fluorescence quenching has been used to monitor the amplification of the target sequences in RT-PCR on a cycle-by-cycle basis.

Quenching in molecular beacons is commonly achieved with the nonfluorescent chromophore, 4-(4′-dimethylaminophenylazo) benzoic acid (DABCYL). Under some circumstances, organic fluorophores are quenched when in very close proximity to metallic surfaces. Lakowicz, J. R., “Radiative Decay Engineering: Biophysical and Biomedical Applications” Anal. Biochem. 2001, 298, 1-24. The presence of metals provides alternative non-radiative energy decay paths that can change the fluorescence quantum yield of a fluorophore. At close distances (<50 angstroms), fluorescence is quenched while at intermediate distances (75 to 100 angstroms), it is enhanced. The phenomena are well documented for Ag and Au films quenching the fluorescence of Rhodamine dyes.

A fluorophore will function and quench appropriately, when linked to an Au surface. See Du, H., Disney, M., Miller, B., and Krauss, T., “Hybridization-Based Unquenching of DNA Hairpins on Au Surfaces: Prototypical “Molecular Beacon” Biosensors” J. Am. Chem. Soc. 2003, 125, 4012-4013. Quenched fluorophore assays on Au colloids can distinguish oligonucleotides with single base mismatches. See Maxwell, D. J., Taylor, J. R., and Nie, S., “Self-assembled nanoparticle probes for recognition and detection of biomolecules” J. Am. Chem. Soc. 2002, 124, 9606-9612; Dubretret, B., Calame, M., and Libchaber, A. J., “Single-mismatch detection using gold-quenched fluorescent oligonucleotides” Nature Biotechnol. 2001, 19, 365-370. This work is possible because fluorescent dyes will reversibly absorb onto colloidal Ag and Au. Nie, S. and Emory, S. R., “Probing Single Molecules and Single Nanoparticles by Surface-Enhanced Raman Scattering” Science 1997, 275, 1102-1106; Krug, J. T., II, Wang, G. D., Emory, S. R., and Nie, S., “Efficient Raman Enhancement and Intermittent Light Emission Observed in Single Gold Nanocrystals” J. Am. Chem. Soc. 1999, 121, 9208-9214. When oligonucleotides are single stranded, they have flexibility and can form looped structures due to their attraction to the Au surface. However, when hybridized, the now double stranded oligonucleotides are rigid such that the fluorescent dye cannot interact with the surface.

There are a number of assays available to interrogate DNA and determine the sequence of bases. These assays range from de novo DNA sequencing of many hundreds of bases at a time to the interrogation of a single base, as in the case of SNP detection. In the majority of these assays, labels are needed to identify a particular product or event from among the thousands of molecules and events also present in the cell or biological extract under interrogation. While there are a few analytical techniques that can directly detect the native molecule, such as mass spectrometry and nuclear magnetic resonance spectroscopy, these often require very specific sample preparation, highly sophisticated and expensive equipment, and often do not work in complex biochemical backgrounds. Therefore, in complex biological systems, the molecule of interest is typically labeled in some way to make it “visible” in order to be assayed. Common labels used in biology include radioactivity, organic fluorophores and quantum dots. However, labeling the molecule being interrogated adds a level of complexity to an assay, thereby making it more difficult to perform properly and consistently, more difficult to turn into a “kit” or product, and more difficult to make the assay field portable and robust due to the additional steps involved. Thus, it would be desirable to have an assay that did not involve a labeling step.

Multiplexing affords the ability to make two or more measurements simultaneously. This has a number of advantages. It reduces the time and cost to collect the measurement. It can often reduce the amount of sample needed to acquire the measurement. More importantly, it allows data to be reliably compared across multiple experiments. Additionally, multiplexing can add confidence to the measurement results through the incorporation of multiple internal controls. Thus, it would be desirable to have an assay that was capable of being used for multiplexed analysis.

Raman scattering is a laser-based optical spectroscopy that, for molecules, generates a fingerprint-like vibrational spectrum with features that are much narrower than fluorescence. Raman scattering can be excited using monochromatic far-red or near-IR light, photon energies too low to excite the inherent background fluorescence in biological samples. Since Raman spectra typically cover vibrational energies from 300-3500 cm⁻¹, one could envisage measuring a dozen (or more) unique Raman active molecules simultaneously, all with a single light source. However, normal Raman is very weak, limiting its utility for use in bioanalytical chemistry. In surface enhanced Raman scattering (SERS), molecules in very close proximity to nanoscale roughness features on noble metal surfaces (gold, silver copper) give rise to million- to trillion-fold increases [known as enhancement factor (EF)] in scattering efficiency. More importantly, SERS can also be used to detect molecules adsorbed to individual metal nanoparticles, and has been used to demonstrate detection of single molecules.

WO 05/019812 to Graham, et al., describes modified molecular beacons detectable by SERS. Recently, Wabuyele and Vo-Dinh have described the use of plasmonics-based nanoprobes that act as molecular sentinels for DNA diagnostics. Wabuyele & Vo-Dinh, (2005) Anal. Chem. ASAP Article; DOI: 10.1021/ac0514671.

### SUMMARY OF THE PRESENT INVENTION

The present invention provides an assay and method of assay for optical detection of bioagents, a target nucleic acid or a target protein using a surface enhanced Raman scattering (SERS) active biomolecule molecular beacon. The present invention also provides the assay and method in a multiplexed format.

### BRIEF DESCRIPTION OF THE FIGURES

FIG. 1 shows a cartoon of the SERS molecular beacon assay in which an oligonucleotide with a Raman reporter molecule on one end (the SERS molecular beacon) is attached to a roughened metal surface. The same hairpin-loop structure is employed, forcing the Raman reporter molecule in contact with the surface, leading to an enhanced Raman signal. Upon hybridization of the oligonucleotide, the Raman reporter molecule is moved away from the surface and the Raman signal is essentially eliminated.

FIG. 2 shows Raman spectra acquired from 50 nm colloidal gold coated with Cy5 molecular beacons after addition of MgCl2 and after further addition of the proper target sequence. Spectra were acquired using 785 nm excitation on a Renishaw in Via Raman microscope using 100% laser power, a 1 second integration time and a 5× objective. Spectra have not been corrected for dilution of the sample after addition of target (˜15% dilution).

FIG. 3 shows Raman spectra acquired from 70 nm colloidal gold coated with Cy5 molecular beacons after addition of varying amounts of NaCl. Spectra were acquired using 785 nm excitation on a Renishaw in Via Raman microscope using 100% laser power, a 1 second integration time and a 5× objective. Spectra have been offset for clarity.

FIG. 4 shows Raman spectra acquired from 70 nm colloidal gold coated with Cy5 molecular beacons and incubated with 80 mM NaCl before and after addition of the target sequence (1 μM final concentration). Spectra were acquired using 785 nm excitation on a Renishaw in Via Raman microscope using 100% laser power, a 1 second integration time and a 5× objective.

FIG. 5 shows a cartoon of the SERS molecular beacon assay using nanowires, and fluorescent beacons.

FIG. 6 shows a comparison of SERS spectra from (A) Cy5 labeled oligonucleotide assembled onto a nanowires, (B) Free Cy5 dye assembled onto a nanowire.

FIG. 7 shows Comparison of SERS spectra from free Cy5 dye assembled onto (A) a nanowire of sequence 111111, all silver, (B) a nanowire of sequence 000001, mostly gold, and (C) 50 nm gold colloid.

FIG. 8 shows an HCV probe assembled onto nanowires and hybridized with and without target sequence. Controls with no target present show (A) Reflectance and fluorescence image pair showing location of nanowires, and lack of fluorescence. (B) SERS spectra observed. Experiment with HCV target sequence hybridized shows (C) reflectance and fluorescence image pair showing location of nanowires, and large fluorescent signal (D) no SERS spectra observed.

FIG. 9 shows a comparison of SERS activity for HCV probe assembled nanowires with no target (control), incorrect target (SARS) and correct target (HCV target) added to hybridization buffer. RRU=relative Raman units (arbitrary).

FIG. 10 shows an HCV target titration study comparing SERS and fluorescent intensity.

FIG. 11 shows a SERS beacon activity using PCR amplicons as target material

FIG. 12 shows a comparison of SERS spectra from (A) free BPE assembled onto nanowires, and (B) free Cy5 dye assembled onto nanowires. All nanowires of sequence 0101010.

FIG. 13 shows an Aptamer Beacon-Based Assay. Comparison of fluorescence intensity aptamer molecular beacons assembled onto nanowires with no target (control), and correct target (thrombin) added to hybridization buffer.

FIG. 14 shows a comparison of sequences for aptamer beacon probes, where THR Apt 1, THR Apt 2 and THR Apt 3 are different hairpin sequence, but same oligonucleotide probe sequences.

FIG. 15 shows a Thrombin Aptamer Beacon Titration study performed in (A) in buffer, and (B) in 50% serum.

FIG. 16 shows specificity of aptamer molecular beacon assay, using alpha-Thrombin specific probes for detection of alpha-Thrombin, beta-thrombin, and ovalbumin, plus blank as negative control.

FIG. 17 shows a schematic of a simple multiplexed assay (two-plex) used to differentiate between two different pathogens.

FIG. 18 shows a schematic of a methods used to verify successful hybridization carried out by using a labeled target nucleic acid, such as a fluorescently labeled target nucleic acid.

FIG. 19 shows the result of a successful linkage of a “pre-hybridized” oligonucleotide to the surface resulting in fluorescence (A), and a miscoupling resulting in a SERS signal (B). FIG. 19 also shows result of successful coupling of an unlabelled thiol-linked probe which is little or no SERS signal (C), and a miscoupling resulting in a SERS signal (D).

### DETAILED DESCRIPTION OF INVENTION

The present invention provides a simple assay that can be performed on non-specialized equipment. The assay may be run in a multiplexed format. The assay has utility with respect to a number of fields, including pathogen monitoring, environmental monitoring, healthcare diagnostics, bio- and chemical terrorism and in field food-borne pathogen detection. The present invention allows “label-free,” multiplexable biomolecule analysis assays and does not require a dedicated and specialized instrument for analysis. The present invention enables a larger number of analyses to be performed faster, in non-laboratory based environments and by non-technical operators. In addition, the assay has high specificity and sensitivity.

It is to be noted that the term “a” or “an” entity refers to one or more of that entity; for example, a protein refers to one or more proteins or at least one protein. As such, the terms “a” (or “an”), “one or more” and “at least one” can be used interchangeably herein. It is also to be noted that the terms “comprising”, “including”, and “having” can be used interchangeably.

In a typical embodiment of the method of the present invention, a SERS-active metal surface is associated with an oligonucleotide, and the oligonucleotide is associated with a Raman-active reporter molecule. An oligonucleotide associated with a Raman-active reporter molecule is sometimes referred to herein as a “SERS molecular beacon” or a “SERS beacon.” When an oligonucleotide with a hairpin-loop structure is employed, the Raman reporter molecule is in contact with the surface, leading to an enhanced Raman signal when excited by a suitable light source. In the looped configuration, the SERS molecular beacon system is in its “on” configuration (as opposed to a fluorescent system which in this configuration would be “off” because the fluorophore is quenched) since a SERS sandwich structure exists. When a bioagent, such as DNA, protein or other target polynucleotide or oligonucleotide) hybridizes to this loop structure, the sandwich configuration is lost and the molecular beacon is in its “off” state. The resulting hybrid is comparatively rigid and causes the Raman reporter molecule to be moved away from the surface and the Raman signal is essentially eliminated. An assay built on the phenomenon of molecular beacons does not require the interrogated nucleic acid to be labeled, nor does it have to be separated from the other components of the assay. There are a number of differences between Raman and fluorescence for the detection of bioagents. With Raman, multiplexed detection is possible due to the ability to vary the Raman reporter molecule, the detection can be performed using IR excitation and therefore is difficult for a third party to detect, and the detection can be achieved from a distance.

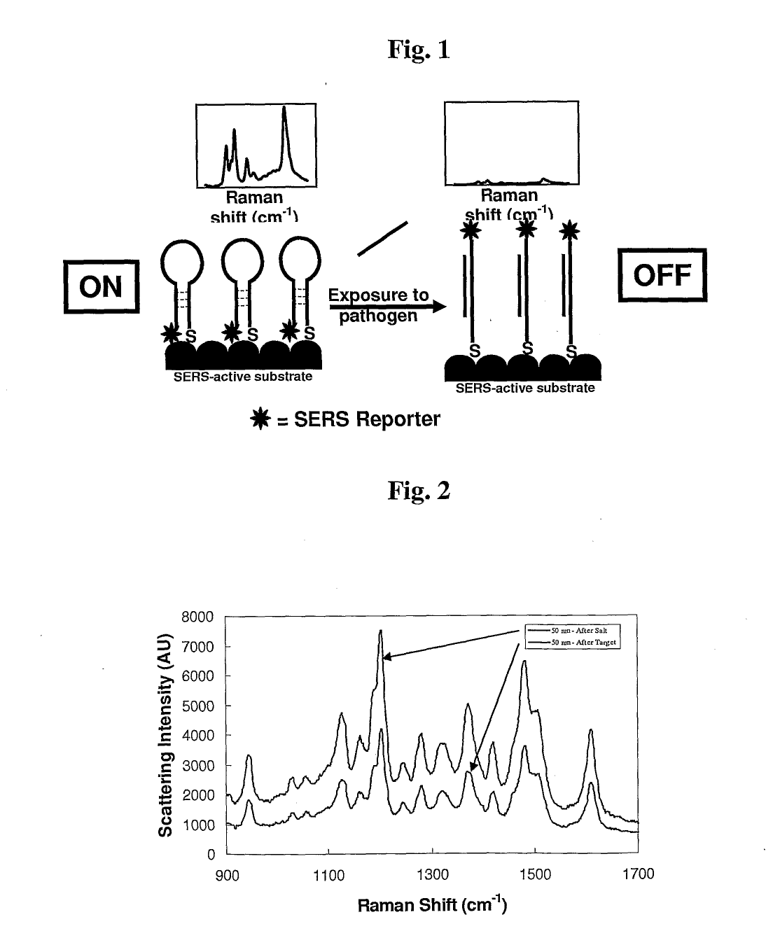

In one embodiment of the invention, the method comprises contacting the target nucleic acid with the SERS-active metal surface associated with oligonucleotide under conditions permitting hybridization; and detecting hybridization. FIG. 1 shows a cartoon of a SERS molecular beacon assay.

Examples of Raman-active reporters suitable for use in the present invention include 4-mercaptopyridine (4-MP); trans-4,4′ bis(pyridyl)ethylene (BPE); quinolinethiol; 4,4′-dipyridyl, 1,4-phenyldiisocyanide; mercaptobenzamidazole; 4-cyanopyridine; 1′,3,3,3′,3′-hexamethylindotricarbocyanine iodide; 3,3′-diethyltiatricarbocyanine; malachite green isothiocyanate; bis-(pyridyl)acetylenes; Bodipy; and isotopes of the foregoing, such as deuterated BPE, deuterated 4,4′-dipyridyl, and deuterated bis-(pyridyl)acetylenes; as well as pyridine, pyridine-d5 (deuterated pyridine), and pyridine-¹⁵N.

As used herein, the term “oligonucleotides” refers to a short polymer composed of deoxyribonucleotides, ribonucleotides or any combination thereof. These oligonucleotides are at least 5 nucleotides in length, but may be about 20 to about 100 nucleotides long. In certain embodiments, the oligonucleotides are joined together with a detectable label, which includes a Raman-active reporter. Oligoncleotides used according to this invention comprise at least a single-stranded nucleic acid sequence that is complementary to a desired target polynucleotide or oligonucleotide (either or both of which shall be referred to herein as a “target nucleic acid”), and a detectable label for generating a signal. Some oligonucleotides include complementary nucleic acid sequences, or “arms,” that reversibly interact by hybridizing to one another under the conditions of detection when the target complement sequence is not bound to the target. In some cases, these oligonucleotides are referred to as “hairpin” oligonucleotides. Hairpin oligonucleotides are described elsewhere in this disclosure. When the detectable label is a Raman-active reporter, the oligonucleotide may be (or function in a similar fashion to) a molecular beacon. Molecular beacons typically comprise a single-stranded oligonucleotide hybridization probes that form a stem-and-loop (hairpin) structure.

The oligonucleotide used need not be a hairpin oligonucleotide. Because single-stranded DNA has a flexible backbone, the DNA is conformationally flexible. Previous studies have shown the many Raman-active molecules spontaneously adsorb on gold and silver surfaces. Additionally, fluorescent beacons have been shown on colloid that do not have hairpins, see Maxwell et al (2002) JACS, 124, 9606. In this case then, oligonucleotides may be conjugated to a metal particle or surface on one end, and have a SERS-active particle or tag in close proximity to the surface of the metal particle or surface on the other end, and where the DNA does not contact the surface of the metal, but rather forms an archlike structure. Both the hairpin (“stem-and-loop”) configuration and non-hairpin (“arched”) configuration are within the scope of the present invention.

The Raman reporter may be associated with the oligonucleotide by method known in the art. The association may be covalent or noncolvalent. In some cases, the reporter is coupled to the 5′- or 3′-end of the oligonucleotide, optionally via a spacer molecule. In other cases, the Raman reporter is associated with the oligonucleotide via a coupling with a base or backbone atom, optionally via a spacer molecule.

Conjugation (linking) of reporter molecules can be effected in several ways. A Raman reporter-functionalized oligonucleotide of the invention can be prepared by conjugation of the reporter to the oligonucleotide using EDC/sulfo-NHS (i.e., 1-ethyl-3 (3-dimethylaminopropylcarbodiimide/N-hydroxysulfosuccinimide) to conjugate the carboxyl end of the reporter with an amino function of the linking group on a nucleotide. Also, a reporter linked oligonucleotide sequence can be prepared by conjugation of the reporter the oligonucleotide via a heterobifunctional linker such as m-maleimido-benzoyl-N-hydroxysulfosuccinimide ester (MBS) or succinimidyl 4-(N-maleimido-methyl)cyclohexane-1-carboxylate (SMCC) to link a thiol function on the reporter to the amino function of the linking group on oligonucleotide. By this mechanism, an oligonucleoside-maleimide conjugate is formed by reaction of the amino group of the linker on the linked nucleosides with the MBS or SMCC maleimide linker. The conjugate is then reacted with molecules having free sulfhydryl groups. A reporter-functionalized oligonucleotide can also be prepared by conjugation of the reporter to the sequence using a homobifunctional linker such as disuccinimidyl suberate (DSS) to link an amino function on reporter to the amino group of a linker on the sequence. By this mechanism, an oligonucleoside-succinimidyl conjugate is formed by reaction of the amino group of the linker on the nucleoside sequence with a disuccinimidyl suberate linker. The disuccinimidyl suberate linker couples with the amine linker on the sequence to extend the size of the linker. The extended linker is then reacted with amine groups. Other chemistries for derivatizing oligonucleotides with reporter molecules are known to those skilled in the art.

The oligonucleotide-conjugated metal particles of the present invention have many applications. They can be used in situations in which ordinary molecular beacons have been used, such as in real-time PCR detection; single-nucleotide mutation screening; allelic discrimination, that is, differentiation between homozygotes and heterozygotes; diagnostic clinical assays in which the oligonucleotide-conjugated encoded metal particles, in conjunction with PCR, can be used to detect the presence and abundance of, for example, certain viruses or bacteria in a tissue or blood sample. These methods are well-known to those of ordinary skill in the art.

The SERS-active surface can be a metal surface where the metal is SERS-active, including a roughened metal surface such as a roughened Ag or Au surface, or a metal nanoparticle, such as an Ag or Au nanoparticle. The surface may also be a surface having isolated metal particles adsorbed to a flat substrate. This includes nanowires, such as Nanobarcodes® particles. Creation of a SERS substrate by the deposition of metal nanoparticles, such as Au nanoparticles, on a clean flat surface has several attractive features. The size of the surface features can be controlled simply by controlling the size of the Au colloid. Spacing between particles can be controlled as has been shown previously. Spacing is important because the interparticle coupling can contribute to SERS enhancement. The spacing is also important to avoid the possibility of a false “negative” signal.

SERS-active surfaces comprising larger and more closely-spaced features may be prepared by electroless deposition of metal. It has been demonstrated that highly SERS-active surfaces can be formed by slow, careful hydroxylamine-mediated reduction of Au³⁺ on surface-confined particles. The beauty of the method lies in the fact that no new particles are formed, insofar as all reduction takes place on the surface of existing particles. Thus, it is possible to prepare a well-defined surface with well-defined interparticle spacing, and measure the SERS response. Then, metal can be deposited incrementally, and the SERS response measured. All that will change will be particle size and interparticle spacing, and in a well-defined and quantifiable fashion.

The SERS-active surface may also be a metal nanoparticle. A solution-based synthesis of 45-nm diameter spherical gold (Au) particles has been found to be reproducible, easy to implement, and to give a reasonably narrow distribution of particle size and shape, leading to reproducible tag formation. By varying the reaction conditions, one can shift the average size of the particles in 10 nm increments up to 90 nm. This can be done by either reducing the number of nuclei for particle formation or by increasing the total amount of Au in the reaction solution. This will therefore yield particles of distinct and varying sizes for investigation.

In other embodiments, the metal nanoparticle includes an additional component, such as in a core-shell particle. Au₂S/Au core-shell particles have been reported to have widely tunable near-IR optical resonance. (Averitt, et al., October 1999, JOSA B, Volume 16, Issue 10, 1824-1832.) Alternatively, Ag core/Au shell particles, like those described in J. Am. Chem. Soc. 2001, 123, 7961, or Au core/Ag shell particles, or any core-shell combination involving SERS-active metals, can be used. Other combinations suitable for use in core-shell particles are included in this invention, such as Au- or Ag-nanoparticle functionalized silica/alumina colloids, Au- or Ag-functionalized TiO₂ colloids, Au nanoparticle capped-Au nanoparticles (see, for example, Mucic, et al., J. Am. Chem. Soc. 1998, 120, 12674), Au nanoparticle-capped TiO₂ colloids, particles having and Si core with a metal shell (“nanoshells”), such as silver-capped SiO₂ colloids or gold-capped SiO₂ colloids. (See, e.g. Jackson, et al., 2004 Proc Natl Acad Sci USA. 101(52):17930-5). Hollow nanoparticles such as hollow nanospheres and hollow nanocrystals may also be utilized as a SERS-active surface.

The SERS-active nanoparticles may be isotropic or anisotropic. Nanoparticles include colloidal metal hollow or filled nanobars, magnetic, paramagnetic, conductive or insulating nanoparticles, synthetic particles, hydrogels (colloids or bars), and the like. It will be appreciated by one of ordinary skill in the art that nanoparticles can exist in a variety of shapes, including but not limited to spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. Another class of nanoparticles that has been described is one with internal surface area. These include hollow particles and porous or semi-porous particles. Moreover, it is understood that methods to prepare particles of these shapes, and in certain cases to prepare SERS-active particles of these shapes, have been described in the literature. While it is recognized that particle shape and aspect ratio can affect the physical, optical, and electronic characteristics of nanoparticles, the specific shape, aspect ratio, or presence/absence of internal surface area does not bear on the qualification of a particle as a nanoparticle.

Much of the SERS literature (both experimental and theoretical) suggests that anisotropic particles (rods, triangles, prisms) may provide increased enhancement compared to spheres. For example, the so-called “antenna effect” predicts that Raman enhancement is expected to be larger at areas of higher curvature. Many reports of anisotropic particles have been recently described, including Ag prisms and “branched” Au particles. The use of such anisotropic particles as a SERS-active surface are within the scope of the invention.

In a multiplexed embodiment of the method, each SERS-active surface, whether a nanoparticle, metal island, surface-deposited nanoparticle, and so on, is conjugated with a different oligonucleotide, each oligonucleotide being associated with a particular reporter molecule. The oligonucleotides may be associated with the metal via a thiol linkage. A record is kept of which oligonucleotide probe is attached to which reporter molecule. Decoding of the “flavor” of the diminished SERS-spectrum indicates which DNA sequence was present.

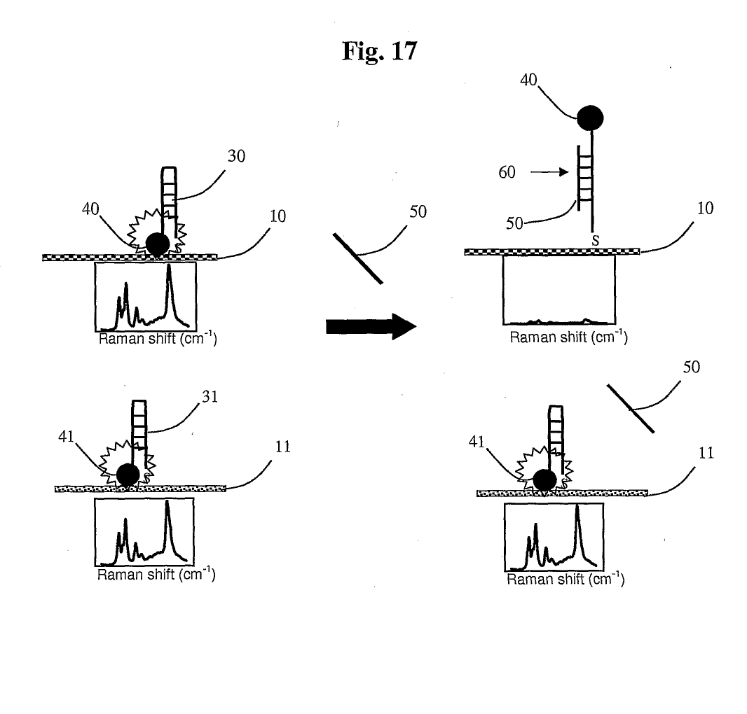

A simple multiplexed assay (two-plex) may be used to differentiate between two different biomolecules. Referring to FIG. 17, two SERS-active surfaces having differing Raman-active reporter molecules are employed to differentiate between Pathogen A and Pathogen B. The first surface 10 is conjugated to the first probe oligonucleotide 30, complementary to DNA from Pathogen A. The second surface 11 is conjugated to the second probe oligonucleotide 31, complementary to DNA from Pathogen B. The probe oligonucleotides are labeled with a Raman reporter molecule at a distance from the attachment to the particle. The first probe oligonucleotide is labeled with a first Raman reporter 40 and the second probe oligonucleotide is labeled with a second Raman reporter 41. The first and second Raman reporters typically are different.

Upon addition of DNA 50 from Pathogen A, hybridization between the pathogen DNA and the complementary sequence 30 occurs. The resulting DNA structure 60 is rigid and therefore causes the Raman reporter 40 to be moved away from the SERS-active surface 20 of the first particle 10. Upon analysis with a Raman-based microscope or other Raman detection instrument, one Raman spectrum will appear bright due to the SERS signal, while the other Raman spectrum will disappear. The Raman spectrum that has been eliminated may be discerned. In this way, the oligonucleotide that hybridized to the Pathogen will be identified as the first oligonucleotide 30. The very large number of possible Raman reporters allows for very high multiplexing without the need to label target nucleic acids.

A number of different configurations could possibly occur when attempting to couple fluorescent oligonucleotides to a SERS-active surface. Distinguishing a successful configuration shown with an unsuccessful configuration that appears “on” presents a challenge for quality control. However, a number of approaches may be used to address this problem. For example, a number of methods may be used to monitor progress of the coupling of the oligos to the SERS-active surface. For example, the oligonucleotides may be displaced from the surface of the particle using mercaptoethanol or other thiol containing molecules via an exchange reaction. Detailed protocols for displacement of thiol-derivatized oligonucleotides from Au colloids and films are available to one of ordinary skill in the art. These methods may be optimized for SERS-active surfaces by carrying out time and temperature course evaluations for a series of mercaptoethanol concentrations to determine the end point of the reaction.

An alternative approach for verifying the successful attachment of the oligonucleotide to the surface uses “pre-hybridized” oligonucleotides, i.e., probe oligonucleotides that already have been hybridized to a complementary sequence prior to being attached to the particle surface. The double-stranded oligonucleotides have more rigidity and so in a successfully attached conformation, there will be little or no SERS signal. Accordingly, a successful linkage to the surface will result in fluorescence. See FIG. 19A. However, in a miscoupling will result in a SERS signal as show in FIG. 19B.

Another alternative approach for verifying successful attachment of the oligonucleotide to the surface is (a) to couple unlabelled thiol-linked probe oligonucleotides to the surface, and then (b) to hybridize the probe oligonucleotides with complementary oligonucleotides that have been fluorescently labeled. A successful coupling followed by successful hybridization will result in little or no SERS signal as shown in FIG. 19C. However, a miscoupling followed by hybridization would result in a SERS signal as shown in FIG. 19D.

As described above, the present invention provides an assay in which a Raman spectrum intensity decreases upon hybridization and Raman spectrum intensity remains unchanged in a negative control experiment. Parameters of an individual assay may be optimized by adjusting the buffer conditions, hybridization times, hybridization temperatures, oligonucleotide sequence requirements, thiol-Au bond stability, and number and character of stringency washes. As used herein, stringent hybridization conditions refer to standard hybridization conditions under which nucleic acid molecules, including oligonucleotides, are used to identify molecules having similar nucleic acid sequences. Such standard conditions are disclosed, for example, in Sambrook et al., Molecular Cloning: A Laboratory Manual, Cold Spring Harbor Labs Press (1989). Sambrook et al., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth, J. et al., Anal. Biochem. 138:267-284 (1984); Meinkoth, J. et al., ibid., is incorporated by reference herein in its entirety. In some embodiments, hybridization conditions will permit hybridization of nucleic acid molecules having at least about 80% nucleic acid sequence identity with the nucleic acid molecule being used to probe. In other embodiments, hybridization conditions will permit isolation of nucleic acid molecules having at least about 90% nucleic acid sequence identity with the nucleic acid molecule being used to probe. In other embodiments, hybridization conditions will permit isolation of nucleic acid molecules having at least about 95% nucleic acid sequence identity with the nucleic acid molecule being used to probe.

One of skill in the art will also be informed by the body of work on fundamental studies on the behavior of nanoparticle-biomolecule and surface-biomolecule interactions. For example, a systematic study of hybridization efficiencies of DNA attached to 12 nm Au nanoparticles has been carried out to characterize the effect of space length, concentration, complement length and oligonucleotide length. In addition, a very thorough model has been provided of the behavior of DNA hybridization in the presence of Au nanoparticles (ranging in size from 13 nm to 50 nm) that explains the sharp hybridization transition temperature observed (which is sharper than observed in an untagged DNA duplex).

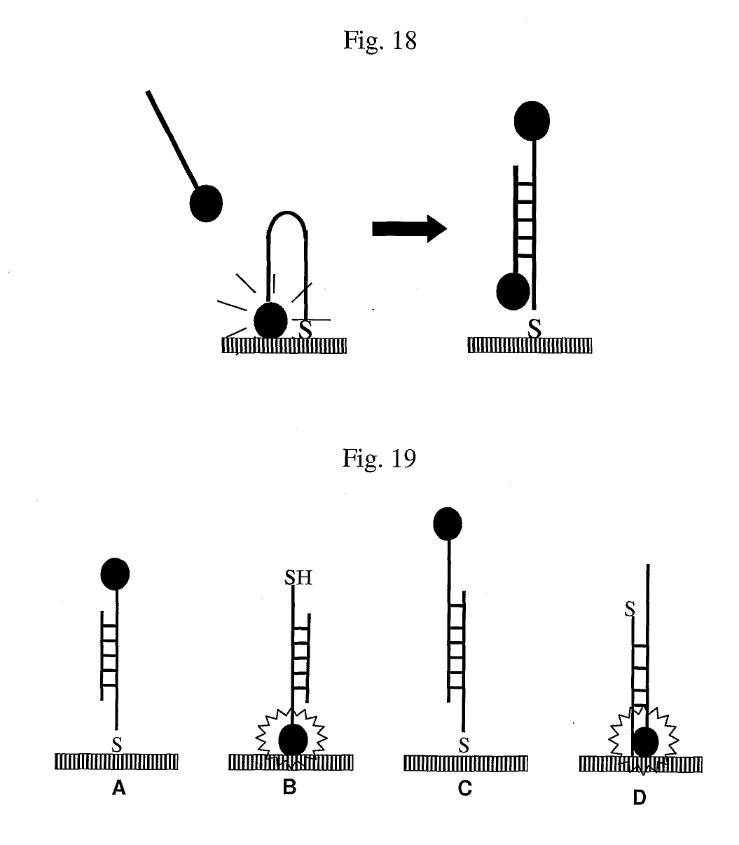

A number of methods may be used to verify successful hybridization. For example, hybridization may be carried out by using a labeled target nucleic acid, such as a fluorescently labeled target nucleic acid, as shown in FIG. 18. The particle-bound probe oligonucleotide is contacted with the labeled target nucleic acid and the labeled nucleotide hybridizes with the probe oligonucleotide. Following hybridization and stringency washes, the fluorescence signal in the reaction is determined. By increasing the temperature and lowering the salt concentration, the double-stranded oligonucleotide may be “melted” to release the labeled target nucleic acid. By centrifuging the reaction and quantitating the fluorescence of the eluent, the amount of oligonucleotides hybridized can be determined. Following this, the oligonucleotides bound on the surface can be displaced with an alkanethiol and the eluent collected and the fluorescence measured. This method will allow the determination of both surface coverage and hybridization efficiency, from the same particles.

The Au-thiol bond is stable under high salt conditions (0.5 M NaCl). Furthermore, the biologically relevant conditions under which the Au-thiol, Ag-thiol and Pt-thiol bonds are stable may be further characterized by determining the effect of varying the temperature from about 25° C. to about 70° C., the effect of varying salt concentration from about 0 to about 1 M, and the effect of the inclusion of about 0 to about 10% SDS detergent and about 0 to about 50% formamide.

In many hybridization assays that occur on a surface, a “spacer” is needed to move the interrogated sequence away from the surface so that the hybridization can occur sterically unhindered. This effect has been reported on planar surfaces, including microarrays, as well as on colloidal Au. The enhancement level of Raman signal from Raman reporter is sensitive to distance from the metal substrate. This distance can be controlled by variation of a conserved DNA sequence in the DNA hairpin-loop structure. The content and length of the sequence may be optimized to maximize the SERS enhancement. The spacer groups may be varied from about 0 to about 20 bases on the nucleic acid sequence, if nucleic acid spacers, and a spacer of the same length, if a hydrocarbon spacer is used. When a spacer is desired, C₆(CH₂)ₓ, may be used. It is important that the length of the spacer (if any) and the oligonucleotide probe are sufficient to allow the Raman reporter to come within the required distance for generating a SERS signal. Longer spacers and oligonucleotide probes are within the scope of the invention, so long as a SERS signal can be generated.

The present invention includes both hairpin configurations and non-hairpin configurations. The use of hairpin sequences, of course, requires internal complementary sequences to form the hairpin, and thus puts some constraints on the overall sequence of the prove oligonucleotide. See Dubertret et al., 2001. Non-hairpin configurations should result in a SERS signal because the oligonucleotide is flexible and the Raman reporter will tend to reside in close proximity to the positively charged metal surface. See Mawell et al., 2002.

The sequence lengths of the probe oligonucleotides may be any length that permits acceptable robustness and reproducibility. The methods, such as those described above, may be used to determine both hybridization efficiency and the effect of length on surface coverage. However, in particular, the sequences may be of between about 8 and about 100 bases in length. When the assay conditions are optimized multiple experiments may be performed in which a dilution series of a PCR product is assayed, to investigate the linearity, dynamic range and sensitivity of a single component assay with a given detection system.

From the measurements obtained from the optimization strategies outlined above, the theoretical limit of detection of the assay of the invention may be determined.

### EXAMPLES

The following examples are provided for illustrative purposes only and are not intended to limit the scope of the invention.

### Example 1

### SERS Beacon Probe Design

The stem-loop structures of the molecular beacons were designed using the software program MFold. The HCV probe sequence was designed from 5′ UTR region. The sequence was: 5′ thiol (CH₂)₆ gcgag CAT AGT GGT CTG COG AAC CGG TGA ctcgc (CH₂)₇ Cy5-3′ (SEQ ID NO: 1). The HCV target sequence was: TCA CCG GTT CCG CAG ACC ACT ATG (SEQ ID NO: 2). All probes and targets were purchased from BioSource. The HCV viral RNA was ordered from Ambion Diagnostics.

### Example 2

### SERS Molecular Beacons Using Gold Colloid Substrates

A 100 μL aliquot of the Cy5 molecular beacon (Cy5-MB) was prepared in ultrapure water. Next, 250 μL of 50 or 70 nm colloidal gold (0.01% Au by weight) was added to the beacon solution. These were incubated for approximately 6 hours before addition of 5 μL of 2.0 M NaCl. After 30 minutes, another 5 μL of NaCl was added. Another 30 minutes was allowed before excess beacon was purified by centrifugation (˜1500 RCF for 12 minutes, repeated 3 times). Particles were resuspended in TE buffer (10 mM TRIS, 0.1 mM EDTA, pH 7.5).

HCV probe assembled colloids were placed into sample wells on a quartz slide. Each well in the gasket was approximately 2 mm in diameter and depth, and held up to 10 μL of solution. Aliquots (5 μL) of each conjugate were placed into separate wells and their Raman spectra interrogated. No SERS peaks were visible using the maximum laser power setting with a 1 second integration time and a 5× objective. It was surmised that the beacons might be assembled onto the particles, but perhaps were not in the “closed” state required to obtain SERS from the reporter. 1 μL of 25 mM MgCl2 was added to each well to promote hybridization of the stem. Spectra were acquired, but while SERS activity was present, the colloid was aggregated (based on a visual color change from pink to blue). Regardless, a 1 μL of 100 μM target was also added to each well and allowed 5 minutes to hybridize before acquiring spectra again. Spectra from the 50 nm conjugates after MgCl2 addition and after addition of target are shown in FIG. 2. Similar results were found for 70 nm colloid (results not shown). Both samples clearly show decreased SERS signals after addition of target, but the aggregation complicates analysis. It is possible that the samples were still aggregating and that the decreased signal is an artifact of this effect.

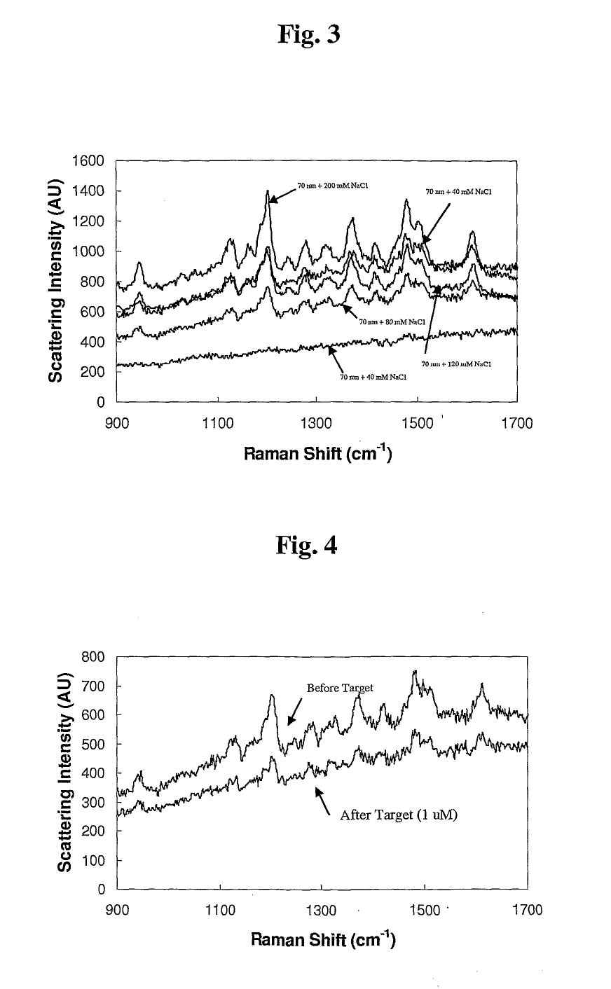

To further investigate the relationship of SERS intensity with the state of aggregation, a series of particles were incubated with varying amounts of NaCl. Samples were prepared that consisted of 9 μL of 70 nm beacon-conjugated particles plus 1 μL of NaCl to give final concentrations of 40, 80, 120, 160 and 200 mM. Spectra are shown in FIG. 3 (offset for clarity). Only the 40 mM sample was not visibly aggregated, and was also the only sample to show no SERS activity. Therefore, it appears as though aggregation is a requirement to observe SERS from molecular beacons on the 70 nm colloidal gold. In spite of this, 1 μL of 100 μM target was added to the 80 mM sample (for a final concentration of ˜20 μM). The target was allowed approximately 10 minutes to hybridize, and the Raman spectrum was acquired. Once again, there is a definite decrease in the Raman scattering after addition of target (FIG. 4), but there are still Cy5 peaks present. It can be likely that some of the targets had not been caught due to a short time hybridization in an un-optimized condition.

### Example 3

### SERS Beacon Assay for Detection of Oligonucleotide Targets, Using Nanowire Substrates

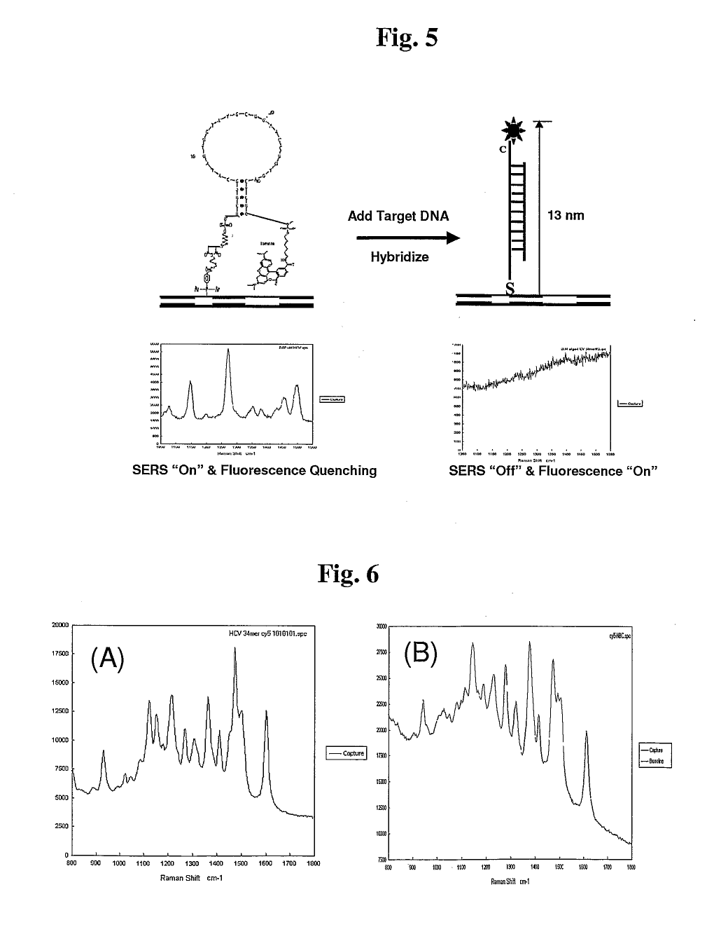

A. Preparation of Nanowires. Gold and silver nanowires (Nanobarcodes® Particles) have been used previously to quench fluorescence based molecular beacons (WO 2005/020890). The advantage of using these substrates it is possible to determine both the SERS response and the fluorescence response, thereby providing an ability to confirm the results using an orthogonal method. FIG. 5 shows a cartoon depiction of this assay format. Nanowires (Nanobarcodes® Particles) were manufactured as previous described (Nicewarner-Pena, S. R., et al., (2001) Science 294, 137-141; Reiss, et al. (2002) J. Electranal. Chem. 522, 95-103; Walton, et al. (2002) Anal. Chem. 74, 2240-2247). Briefly, alternating layers of gold and silver are electroplated into the pores of an alumina template, the template is dissolved using strong base, resulting in the formation of striped nanowires. The nanowires used in this study were 250 nm by 6 μm, and contained 6 metallic segments. 0 denotes a gold segment and 1 denotes a silver segment.

B. Probe Attachment to Nanowires. Oligonucleotide probes were assembled onto the nanowires as follows. Approximately 10⁸ nanowires in 100 μl water, were washed twice with 10 mM PBS, and resuspended in 100 μl 10 mM PBS. Next 500 μl of 5 μM oligonucleotide probes was added and allowed to self-assemble overnight at room temperature, with gentle rotation. Following assembly 600 μl of 0.3M NaCl in 10 mM PBS was added, and allowed to react 2 hours in an aging step. The particles were then washed twice in 0.3M NaCl in 10 mM PBS, resuspended in 100 μl 10 mM PBS and stored at 4° C. until ready to use.

C. SERS Characterization Using Nanowire Substrates. HCV probe was assembled onto nanowires of a sequence 101010, in which 1=silver segment and 0=gold segment. After washing twice as described in the methods section, 3 μl of probe conjugated nanowires were images on a quartz slide, using a Reinshaw Raman microscope with 736 nm excitation. FIG. 6A shows the Raman spectra, which was postulated to come from the Cy5 dye on the HCV probe. To confirm this theory, a control experiment was performed in which 5 ul of 1 uM of the free dye Cy5 (as a mono NHS ester) was incubated with 10 μl nanowires of the same sequence, and imaged. FIG. 6B shows that the Raman spectra from the control is indeed the same as from the molecular beacon probe, confirming we are observing the SERS signal from Cy5 dye.

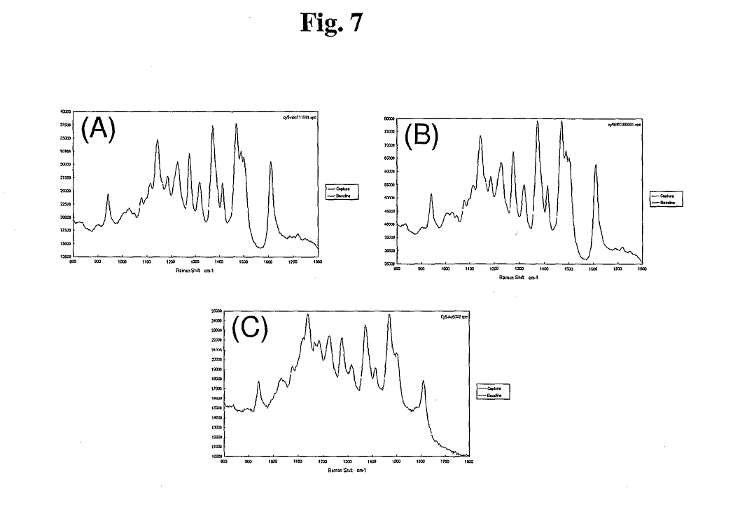

Further characterization was performed to determine whether gold and silver segments of the nanowires had different enhancement abilities. Cy5 free dye was assembled onto nanowires (using same protocols as above) that were all silver (sequence 111111, FIG. 7A), and mostly gold (sequence 000001, FIG. 7B). The Raman spectra are not appreciably changed. A further control was performed using 50 nm Au colloid, and again the spectra show similar peaks (FIG. 7C). With an understanding of the SERS spectra of Cy5, we proceeded to assay development.

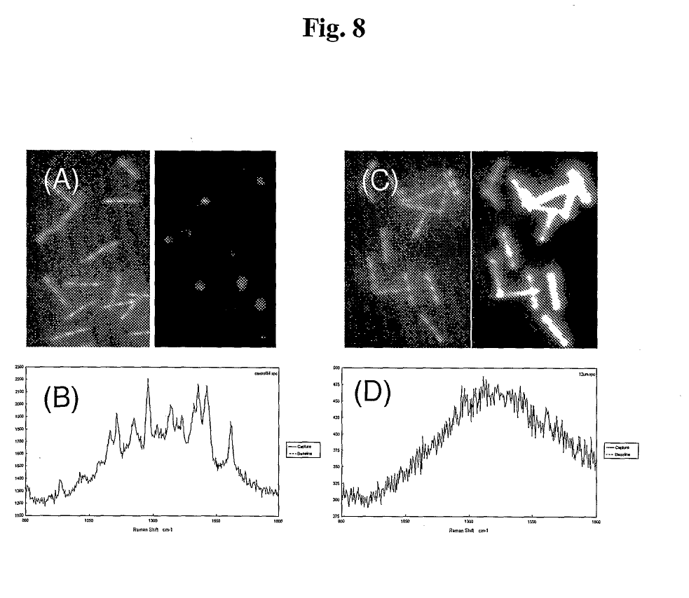

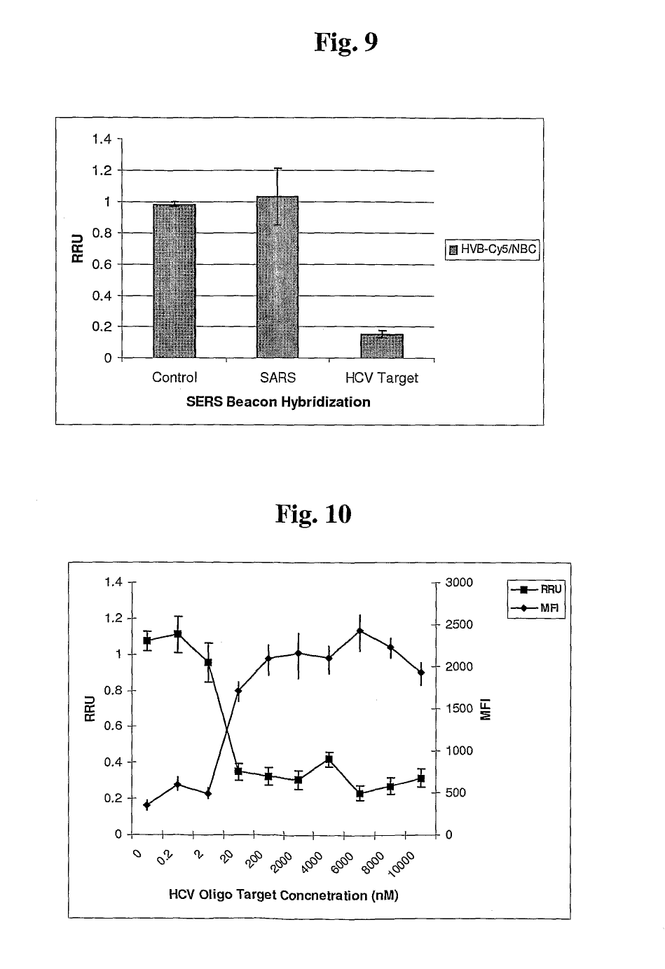

D. SERS Beacon Assay for Detection of Oligonucleotide Targets. HCV probe was assembled onto nanowires of sequence 010101, as above. HCV target sequence (10 μM) was hybridized with one aliquot of probe labeled nanowires, and a second aliquot was used as a negative control in which no target sequence (buffer only) was hybridized, with the nanowires subjected to the same hybridization protocols. FIG. 8B shows the SERS spectra from the negative control, which as expected showed no loss of SERS signal. To confirm this result the nanowires were also imaged on a fluorescence microscope, and no fluorescence signal was observed (FIG. 8A). This is to be expected since in the “closed” orientation the fluorescence from the Cy5 is quenched. Upon addition of 10 μM HCV target sequence, FIG. 8D shows that the SERS signal is significantly reduced. To confirm that this is due to hybridization, a fluorescence image was again taken, and as expected fluorescence was restored (FIG. 8C). FIG. 9 shows the SERS results in graph format, showing that the SERS signal was reduced to 14% of the control signal (no target sequence added) when it was hybridized with 2 μM HCV target. To further confirm that the HCV target sequence was not merely displacing the HCV probe sequence from the nanowire, a control was performed using a target sequence that was not complementary to the probe sequence. A target sequence to the SARS virus was used. As shown in FIG. 9, there was no loss of SERS signal upon addition of this incorrect sequence, thereby confirming we were observing a molecular beacon effect.

E. Titration Data, From Nanowire Substrates. In order to understand the performance of this novel SERS molecular beacon on nanowires, a titration experiment was performed. HCV probe was assembled onto nanowires (0101010) and target added to different aliquots at concentrations ranging from 200 pM to 1000 nM. Both fluorescence and Raman spectra were collected from each aliquot. Data are shown in FIG. 10. The results show that the SERS signal begins to decrease at a concentration of 2 nM target added, and the fluorescence signal begins to increase at <20 nM added target. This further confirms that we are observing a SERS molecular beacon. It is interesting to note that the SERS molecular beacon is 10-fold more sensitive to target concentration than the fluorescent assay, even in this unoptimized format.

F. SERS Beacon Assay for Detection of Real-World Targets, Using Nanowire Substrates.

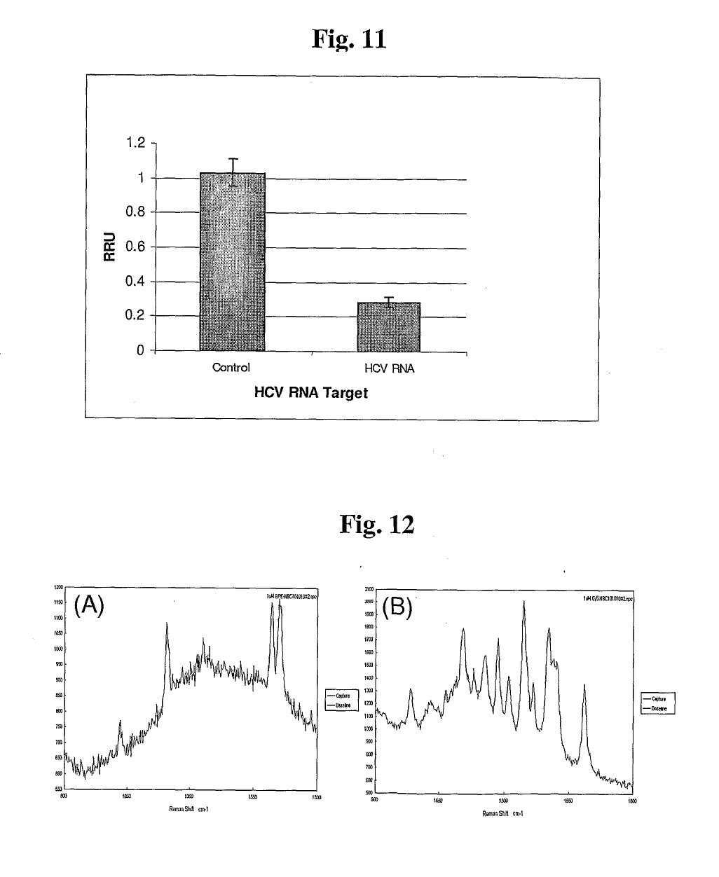

All work performed thus far used synthetic oligonucleotide targets. In order to demonstrate the usefulness of this assay in a real-world application, we investigated the use of RNA derived targets. A sample of RNA from the HCV virus was amplified, using RT-PCR and HCV sequence-specific PCR primers as described in Example 5. The PCR amplicon was hybridized with the HCV conjugated nanowire (55 C for 60 min) as described in Example 4. After stringency washing (1×SSC), the Raman spectra acquired. FIG. 11 shows that the PCR amplicons behave in the same manner as oligonucleotide sequences, with the SERS signal decreased upon hybridization. This is an encouraging result, showing that upon optimization this assay will function for a real-world application.

### Example 4

### Hybridization Assay

Approximately 3×10⁶ nanowires in 3 μl of PBS, were added to 33 μl of hybridization buffer (HS114, Molecular Research Center, Inc), and target in a volume of 16 μL and boiled (to denature PCR sample) for 2 minutes. The reaction was tumbled gently for 1 hour at 55° C. The nanowires were washed with 500 μl 1×SSC for 5 minutes, followed by 500 μl 0.1×SSC for 5 minutes. The particles were resuspended in 8 μl 5 mM PBS, loaded onto a quartz slide and the Raman signal acquired using a Reinshaw Instrument. Fluorescence measurements were taken using an in-house inverted fluorescence microscope from a 96-well plate.

### Example 5

### RT-PCR and Lambda Exonuclease Digestion

Reactions were performed using the Superscript one-step RT-PCR kit (Invitrogen, CA). 5 μl viral RNA was incubated at 75° C. for 3 min, and added to a mix containing 25 μl 2× reaction buffer, 1 μl 10 μM primer 1 and 1 μl 10 μM primer 2, 1 μl Taq polymerase and 17 μl H2O (to 50 μl total reaction volume). The following conditions were performed on a thermocycler: 50° C. 30 min, 94° C. 2 min, then 40 cycles at 94° C. 15 s, 60° C. 30 s and 72° C. 30 s., and a final 72° C. 10 min and hold at 4° C. The double stranded PCR product was designed with a 5′ phosphate group, such that lambda exonuclease could be used to digest away the phosphorylated 5′ strand, leaving the non-phosphorylated 3′ strand for hybridization to the probe. The reaction was allowed to proceed for 20 minutes at 37° C., and then boiled for 1 minute to inhibit any further enzyme activity. The PCR product was designed to locate the oligonucleotide complementary sequence approximately in the middle of the amplicon. The length of the HCV PCR product was 410 bases.

### Example 6

### Data Collection and Analysis

The Raman spectra was obtained using a Reinshaw Invia microscope with 5× objective, 1 s acquisition time and the spectrometer grating centered at 1300 cm⁻¹ and 785 nm excitation. The data was analysis with SenserSee™ software, an in-house written program.

The fluorescence signal collection was performed on a Zeiss Axiovert 100 microscope fitted with a Prior H107 stage, Sutter Instruments 300 W Xe lamp with liquid light guide, Physik Instrumente 400 micron travel objective positioner and Photometrics CoolSnapHQ camera. Images were acquired with a 63×, 1.4 NA objective. The microscope and all components were controlled by a software package that performs intra and inter well moves, automatically focuses at each new position, acquires a reflectance image of the particles at 405 nm and finally acquires the corresponding fluorescence image. The reflectance and fluorescence image pairs were analyzed by NBSee™ Software, an image analysis software package that identifies the nanowires and quantifies their associated fluorescence.

### Example 7

### Non-Fluorescent SERS Reporter Molecules, Using Nanowire Substrates

A desired task is to prepare hairpin-loop oligonucleotides with SERS-only reporter molecules (i.e. non-fluorescent molecules), and use these for molecular beacon experiments. As a first step, experiments were performed to predict the signal levels we may expect from such non-fluorescent reporters. A commonly used reporter molecule for SERS is bis-pyridylethylene (BPE). Nanowires (6 ul at 10⁹ particle per 1 mL) were incubated with either 4 ul of 1 uM BPE or 1 uM Cy5, for 20 minutes. SERS spectra were collected on the Raman microscope. FIG. 12 shows the spectra from both populations of nanowires. Two observations were made, (i) the signal from the Cy5 was much larger than from BPE, and (ii) the BPE signal appeared to be unstable, falling rapidly during the course of the measurement. There are a number of theories that could explain this data. Firstly, it is possible that we are seeing surface enhanced resonant Raman scattering (SERRS) from the Cy5, leading to a greater signal for Cy5 than BPE. However, this may be unlikely given the excitation maximum for Cy5 is 643 nm, and the emission maximum is 667 nm; both far from the 785 nm laser line used in these experiments. Secondly, while we are adding the same concentration of the two respective reporter molecules in with the nanowires, it is possible that Cy5 has a greater affinity for the surface than does BPE. More Cy5 on the surface of the nanowires would lead to greater signal, regardless of the per molecule enhancement factor. This may also explain the drop in signal from BPE during the course of the experiment, as the local heating drives the less strongly adsorbed BPE from the surface.

### Example 8

### Aptamer Molecular Beacon Protocols using Nanowire Substrates

As a first step toward a SERS beacon assay for protein detection, we have developed a fluorescence based aptamer molecular beacon using the nanowire substrates. Aptamers are DNA or RNA sequences with an ability to bind nucleic acid, proteins, small organic compounds, and even entire organisms. We postulated that a molecular beacon designed to bind proteins should function as a DNA:DNA beacon.

The probe designs were as follows:

THR Apt1:

(SEQ ID NO: 3)

5′ thiol (CH₂)₆ CCAACGGTTGGTGTGGTTGG (CH₂)₇
TAMRA -3′.
THR Apt2:

(SEQ ID NO: 4)

5′ thiol (CH₂)₆ gcgagGGTTGGTGTGGTTGGctcgc (CH₂)₇
TAMRA -3′.
THR-Apt3:

(SEQ ID NO: 5)

5′ thiol (CH₂)₆ TGGTTGGTGTGGTTGG (CH₂)₇ TAMRA -3′.
Target sequence:

(SEQ ID NO: 6)

THR apt1-T: CCAACCACACCAACC.

Probe assembly was performed as for standard molecular beacons. The assay was performed by diluted the thrombin protein with Tris-HCl buffer to attain desired concentration. Then 50 μl thrombin protein solution was mixed with 3 μl nanowire assembled aptamer probe in a microfuge tube and incubated for 30 min, with rotation at room temperature. The contents were centrifuged, washed with 0.1% Tween-20/PBS once and fluorescence images acquired using fluorescence microscope described above.

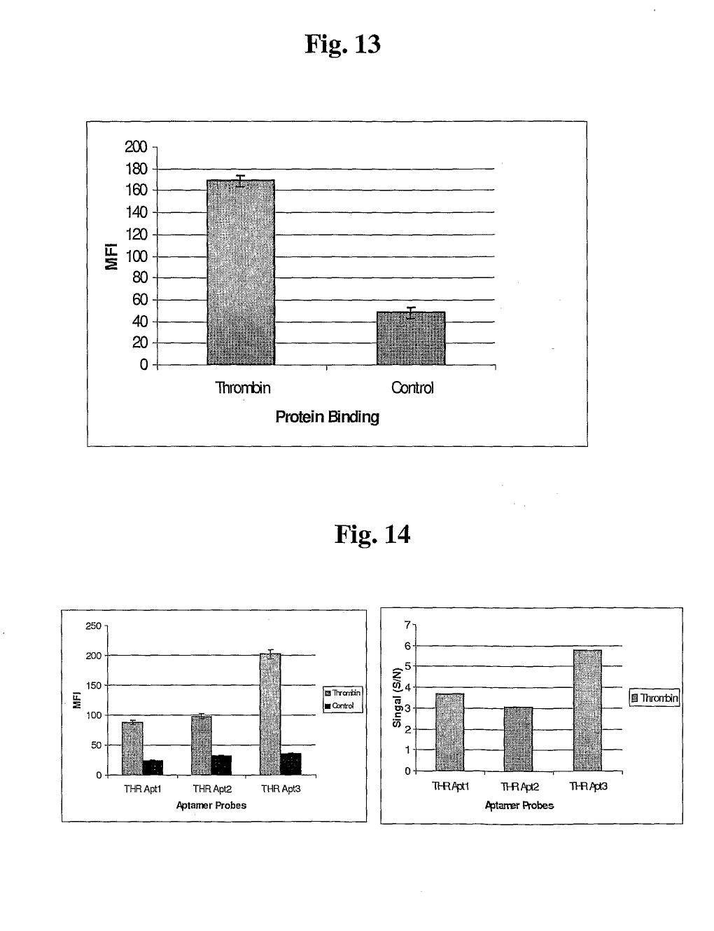

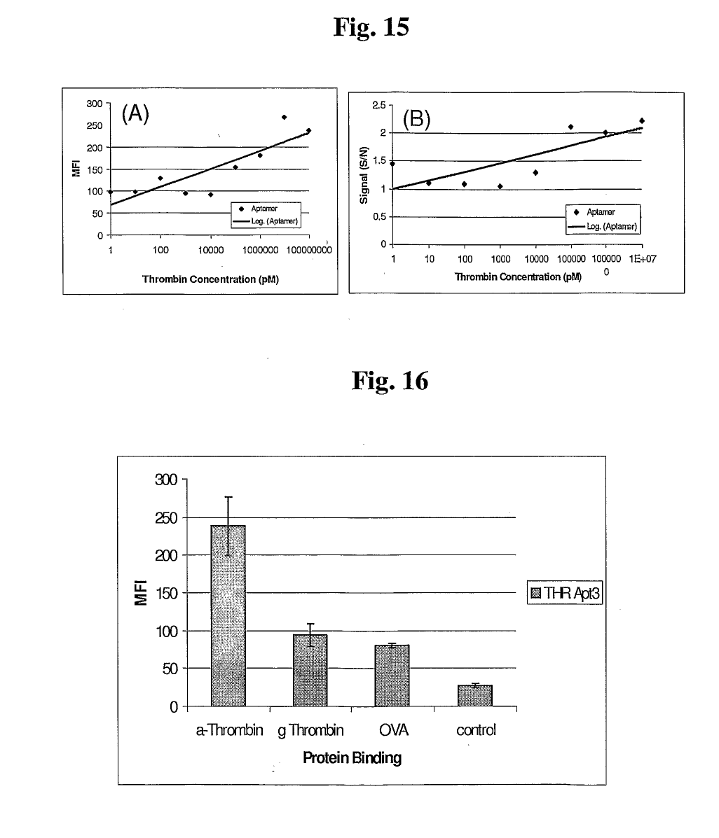

As expected no fluorescence was observed, due to the fluorescence quenching of the TAMRA by the nanowire. However, when the thrombin protein was added to the reaction by incubating 10 μg/ml thrombin with 3 μl nanowire-Apt1 assembled probes for 30 min, fluorescence was restored. The results are shown in FIG. 13. An investigation into the effect of the hairpin in the beacons was carried out. Two additional probes were designed, THR Apt 2 which contained a hairpin sequence, and THR Apt 3 which did not contain a hairpin sequence. Following hybridization, the sequence without the hairpin gave the highest signal to noise ratio, as shown in FIG. 14. This sequence was therefore used for subsequent experiments. FIG. 15A shows data from a titration study showing that thrombin could be detected at 50 nM concentrations in buffer. When this experiment was repeated in 50% serum, the detection limit was again approximately 50 nM (FIG. 15B). These are very encouraging results. Finally, it is important to understand the specificity of the assay, in addition to the sensitivity. THR Apt 3 assembled nanowires were incubated with a pair of homologous proteins, α Thrombin and β Thrombin, and with ovalbumin as a negative control, and a blank (labeled control). As expected, signal was greatest from α Thrombin, following by partial signal from the β homologous thrombin, and little signal from ovalbumin (FIG. 16). This demonstrates that the assay is specific.

What is claimed is:

1. A method for detecting a target nucleic acid, comprising:
a) providing an SERS-active surface comprising an oligonucleotide, said oligonucleotide comprising a Raman reporter molecule;
b) contacting the target nucleic acid with the SERS-active surface comprising an oligonucleotide under conditions permitting hybridization;
c) illuminating the SERS-active surface with light capable of exciting the Raman reporter molecule; and
d) detecting hybridization.

2. The method for detecting a target nucleic acid of claim 1 wherein hybridization is detected by a decrease in a SERS signal.

3. The method for detecting a target nucleic acid of claim 1 wherein said detecting hybridization is performed under stringent hybridization conditions.

4. A method for detecting a plurality of target nucleic acids, comprising:
a) providing a plurality of types of SERS-active surfaces, said surfaces comprising a plurality of oligonucleotides, each of said oligonucleotide comprising a Raman reporter molecule, wherein at least one of said surfaces is differentiable from another of said surfaces based on the Raman reporter molecule;
b) contacting a plurality of target nucleic acids with the plurality of SERS-active surfaces under conditions permitting hybridization;
c) illuminating the SERS-active surfaces with light capable of exciting the Raman reporter molecules;
d) detecting hybridization to the target nucleic acids; and
e) identifying the types of SERS-active surface which exhibits hybridization.

5. The method for detecting a plurality of target nucleic acids of claim 3 wherein the type of SERS-active surface which exhibits hybridization is identified by determining which of the Raman spectrums associated with each of the types of reporter molecule has been decreased.

6. A method for detecting a target protein, comprising:
a) providing an SERS-active surface comprising an aptamer, said aptamer comprising a Raman reporter molecule;
b) contacting the target protein with the SERS-active surface comprising an aptamer under conditions permitting specific binding of the aptamer and its target;
c) illuminating the SERS-active surface with light capable of exciting the Raman reporter molecule; and
d) detecting specific binding of the protein and the aptamer by detecting a decrease in SERS signal.

7. A method for detecting a plurality of target proteins, comprising:
a) providing a plurality of SERS-active surfaces comprising one or more types of SERS-active surfaces, said surfaces comprising a plurality of aptamers, each of said aptamers comprising a Raman reporter molecule, wherein at least one of said surfaces is differentiable from another of said types based on the Raman reporter molecule;
b) contacting a plurality of target proteins with the plurality of SERS-active surfaces under conditions permitting hybridization;
c) illuminating the SERS-active surfaces with light capable of exciting the Raman reporter molecule;
d) detecting specific binding of the proteins and the aptamers; and
e) identifying the type of SERS-active surface which exhibits specific binding.

8. A nucleic acid assay comprising a substrate having isolated SERS-active particles adsorbed thereon the SERS-active particles comprising an oligonucleotide, said oligonucleotide comprising a Raman reporter molecule.

9. The nucleic acid assay of claim 8 wherein the spacing between the SERS-active particles on the substrate is controlled to achieve interparticle coupling and contribute to enhancement of a SERS signal.

10. The nucleic acid assay of claim 8 wherein the SERS-active particle is a core-shell particle having tunable near-IR optical response.

11. The nucleic acid assay of claim 8 wherein the SERS-active particle is a hollow particle.

12. A nucleic acid assay comprising a SERS-active surface comprising an oligonucleotide, said oligonucleotide comprising a Raman reporter molecule, wherein the SERS-active surface is a core-shell particle.

13. A nucleic acid assay comprising a SERS-active surface comprising an oligonucleotide, said oligonucleotide comprising a Raman reporter molecule, wherein the SERS-active surface is a hollow particle.


SEQUENCE LISTING (6 sequences)

SEQ ID NO: 1 - DNA, 34 bp, Artificial
  artificial sequence probe
gcgagcatagtggtctgcggaaccggtgactcgc

SEQ ID NO: 2 - DNA, 24 bp, Artificial
  artificial sequence probe
tcaccggttccgcagaccactatg

SEQ ID NO: 3 - DNA, 20 bp, Artificial
  artificial sequence probe
ccaacggttggtgtggttgg

SEQ ID NO: 4 - DNA, 25 bp, Artificial
  artificial sequence probe
gcgagggttggtgtggttggctcgc

SEQ ID NO: 5 - DNA, 16 bp, Artificial
  artificial sequence probe
tggttggtgtggttgg

SEQ ID NO: 6 - DNA, 15 bp, Artificial
  artificial sequence probe
ccaaccacaccaacc